(19) United States
(12) Patent Application Publication
(10) Pub. No.: US 2024/0399025 A1
(43) Pub. Date: Dec. 5, 2024

(54) BIOACTIVE IMPLANTS AND METHODS OF MAKING AND USE

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Nehal N. Patel, Boston, MA (US); Timothy Young, Upton, MA (US); Justin A. Barbas, West Bridgewater, MA (US); Matthew D. Cunningham, Lakeville, MA (US); Jon-Paul Rogers, North Smithfield, RI (US); Geoffrey I. Karasic, Raynham, MA (US); Stephen A. Santangelo, Tolland, CT (US); William Middleton, Middleton, MA (US); Hadi El Heneidi, Watertown, MA (US); Angela Noble, Chelmsford, MA (US); Mark E. Housman, North Attleboro, MA (US); Marc J. Balboa, Hopkinton, MA (US); Andrew F. Thompson, York (GB); Rosanne Frederick, Mansfield, TX (US)

(73) Assignee: Smith & Nephew, Inc., Memphis, TN (US)

(21) Appl. No.: 18/675,827

(22) Filed: May 28, 2024

Related U.S. Application Data
(60) Provisional application No. 63/469,905, filed on May 31, 2023.
(60) Provisional application No. 63/540,439, filed on Sep. 26, 2023.

(51) Int. Cl. A61L 27/36; A61F 2/08; A61F 2/30

(52) U.S. Cl. CPC A61L 27/3616; A61F 2/08; A61F 2/30756; A61L 27/3691; A61F 2220/0008

(57) ABSTRACT

Methods of making an enhanced bioactive PRP implant include supplying a centrifuge container with a collagen-based scaffold pre-inserted onto a moveable filter within the vial. A centrifuge process for creating the PRP implant is followed, resulting in a PRP/collagen implant combination. Other biologics may be added to the container instead of or in addition to the collagen-based scaffold. Additionally, methods of fixing the PRP implant into place at the point of a tear or wound include applying the PRP implant adjacent to or within a meniscal tear, or underneath or on top of a rotator cuff tear. Suture or staples may then be routed either through or over the PRP implant to hold the implant in place.

### CROSS-REFERENCE TO RELATED APPLICATIONS

This application claims priority to and benefit of U.S. Provisional Application No. 63/469,905, filed on May 31, 2023, entitled BIOACTIVE IMPLANTS AND METHODS OF MAKING AND USE, and U.S. Provisional Application No. 63/540,439, filed on Sep. 26, 2023, entitled BIOACTIVE IMPLANTS AND METHODS OF MAKING AND USE, the entire contents of which are incorporated herein by reference for all purposes.

### FIELD

The present disclosure relates to bioactive surgical implants, as well as methods of using the implants in a tissue repair. In particular, the disclosure relates to platelet-rich plasma implants used in a meniscal or rotator cuff repair.

### BACKGROUND

Platelet-rich-plasma (PRP) and other orthobiologics used to promote healing in a tissue repair are typically fluidic and nonstructural. Therefore, after implantation into a repair site, it is difficult to ensure that the product stays in place long enough to bring and keep biological benefits to the intended site. One solution is a bioactive surgical implant, such as a “patch” made from fibrin sealants and platelet concentrates, which can be produced by isolating the PRP from anti-coagulated whole blood. Such PRP implants can be used for covering wound or tear surfaces to initiate healing.

To achieve optimal healing with the PRP implant, surgeons often desire to combine other types of biologic products, such as collagen, along with the implant. However, such surgical repairs may become cost prohibitive when using both products. Therefore, it would be advantageous to create bioactive PRP implants that also contain other types of orthobiologics for insertion into a repair site.

Additionally, if the PRP implant is placed into a pocket of tissue that will prevent migration, the PRP implant may be applied to the repair without any secondary fixation. However, in many cases, a surgeon may prefer to fix the PRP implant in a specific location to prevent further movement post-operatively during patient range of motion. Therefore, it would be advantageous to provide methods for fixing the PRP implant in place in a repair to achieve optimal healing.

### SUMMARY

The disclosure describes a method of making an enhanced bioactive PRP implant by supplying a centrifuge container with a collagen-based scaffold pre-inserted onto a moveable filter within the vial. The normal centrifuge process for creating the PRP implant is then followed, resulting in a PRP/collagen implant combination that does not require two separate devices or products.

The disclosure also describes devices and methods that allow various liquid biologics instead of or in addition to the collagen-based scaffold to be centrifuged within the container to create patches for insertion into a repair site. The liquid biologics could include bone marrow aspirate concentrate (BMAC), adipose tissue, hyaluronic acid (HA), placental tissue, stem cells, allografts, other biologic adjuncts, loose collagen, or mixtures thereof. The devices connect a syringe filled with the orthobiologics to the container in a sterile manner. A rubber media barrier restricts media or fluid outflow from the syringe until it is inserted into the vial, at which point the barrier is pushed open and the media can fill the container by depressing the syringe.

The disclosure also describes methods of fixing the PRP implant into place at the point of a tear or wound with suture anchors or staples. In particular, the disclosure describes applying the PRP implant adjacent to or within a meniscal tear. The disclosure also describes applying the PRP implant underneath or on top of a rotator cuff tear. In both types of repair, suture may be routed either through or over the PRP implant to hold the implant in place.

Further examples of the PRP implant and methods of making and use of this disclosure may include one or more of the following, in any suitable combination.

In examples, a method of preparing a bioactive implant for application to a wound or a tear in a tissue of a patient of this disclosure includes injecting a volume of the patient's whole blood into a container. The container includes a collagen-based scaffold disposed within the container and a movable member locked within the container. A centrifugal force is applied to the container sufficient to separate the bioactive implant from a layer of serum and a layer of erythrocytes. The moveable member is then released such that the moveable member moves though the container under the centrifugal force to couple the collagen-based scaffold with the bioactive implant, whereby the bioactive implant is suffused with collagen from the collagen-based scaffold.

In further examples, the moveable member is a filter. In examples, the bioactive implant includes platelet rich plasma. In examples, the collagen-based scaffold further includes one or more biologics selected from a group including bone marrow aspirate concentrate (BMAC), adipose tissue, hyaluronic acid (HA), placental tissue, stem cells, allografts, other biologic adjuncts, loose collagen, or mixtures thereof. In examples, after injecting the volume of the patient's whole blood into the container, the whole blood is allowed to coagulate. In examples, the collagen-based scaffold is derived from highly purified bovine Achilles tendon. In examples, the bioactive implant is removed from the container.

Examples of a bioactive implant of this disclosure include a bioactive implant formed by the methods described above.

Examples of a system for inserting a liquid biologic material into a container of this disclosure include a syringe containing the liquid biologic material. A needle cannula couples to a distal end of the syringe. A retractable barrier couples to a distal end of the needle cannula. A connector couples to the needle cannula such that the retractable barrier extends into an interior of the connector. The connector is configured to be disposed over an opening of a container. A needle in fluid communication with the syringe extends through the needle cannula and into the barrier. When the connector is disposed over the opening in the container, the opening causes the retractable barrier to retract, exposing a distal end of the needle to deliver the liquid biologic material into the container. In further examples, the container comprises a vacuum. In yet further examples, the opening is a V-shaped opening defined through a lid of the container.

Examples of a method of tissue repair of this disclosure include placing a bioactive implant in contact with an area in need of repair in a tissue of a patient. The bioactive implant is secured to an area adjacent the area in need of repair with at least one securing device extending through or around the bioactive implant. The bioactive implant is formed by a process of injecting a volume of the patient's whole blood into a container and applying a centrifugal force to the container sufficient to separate the bioactive implant from a layer of serum and a layer of erythrocytes. In further examples, the bioactive implant is coupled to a collagen-based scaffold. In examples, the tissue is a tendon, ligament or other soft tissue. In further examples, the tissue is a rotator cuff or a meniscus. In examples, the securing device is a suture or a staple. In examples, the area in need of repair is a wound or a tear.

A reading of the following detailed description and a review of the associated drawings will make apparent the advantages of these and other features. Both the foregoing general description and the following detailed description serve as an explanation only and do not restrict aspects of the disclosure as claimed.

### BRIEF DESCRIPTION OF THE DRAWINGS

Reference to the detailed description, combined with the following figures, will make the disclosure more fully understood, wherein:

FIG. 1 illustrates a centrifuge container for creating a bioactive implant, according to an embodiment of this disclosure;

FIG. 2 illustrates another example of the container of FIG. 1 including a compaction member, according to an embodiment of this disclosure;

FIG. 3 illustrates another example of the container of FIG. 1 including a collagen-based scaffold, according to an embodiment of this disclosure;

FIGS. 4 and 5 illustrate a method of creating the bioactive implant during the centrifuge and coagulation process, according to an embodiment of this disclosure;

FIGS. 6A-6C illustrate an example of a system for inserting a liquid biologic material into a centrifuge container, according to an embodiment of this disclosure;

FIG. 7 is a flow-chart illustrating the steps of a method of operating a centrifuge to create the bioactive implant 10 using the system of FIGS. 6A-6C;

FIGS. 8A and 8B illustrate a method of using the bioactive implant a meniscal repair, according to an embodiment of this disclosure;

FIGS. 9A and 9B illustrate an alternative method of using the bioactive implant a meniscal repair, according to an embodiment of this disclosure;

FIGS. 10A and 10B illustrate a method of using the bioactive implant a rotator cuff repair, according to an embodiment of this disclosure;

FIG. 11 illustrates the use of a tissue grasper with the bioactive implant, according to an embodiment of this disclosure; and

FIGS. 12A and 12B illustrate the use of a meniscal repair device with the bioactive implant, according to an embodiment of this disclosure.

### DETAILED DESCRIPTION

In the following description, like components have the same reference numerals, regardless of different illustrated examples. To illustrate examples clearly and concisely, the drawings may not necessarily reflect appropriate scale and may have certain features shown in somewhat schematic form. The disclosure may describe and/or illustrate features in one example, and in the same way or in a similar way in one or more other examples, and/or combined with or instead of the features of the other examples.

In the specification and claims, for the purposes of describing and defining the invention, the terms “about” and “substantially” represent the inherent degree of uncertainty attributed to any quantitative comparison, value, measurement, or other representation. The terms “about” and “substantially” moreover represent the degree by which a quantitative representation may vary from a stated reference without resulting in a change in the basic function of the subject matter at issue. Open-ended terms, such as “comprise,” “include,” and/or plural forms of each, include the listed parts and can include additional parts not listed, while terms such as “and/or” include one or more of the listed parts and combinations of the listed parts. Use of the terms “top,” “bottom,” “above,” “below” and the like helps only in the clear description of the disclosure and does not limit the structure, positioning and/or operation of the disclosure in any manner.

FIG. 1 illustrates a centrifuge container 1 for creating the platelet-rich bioactive implant 10, according to an embodiment of this disclosure. As shown in FIG. 1, the container 1 may comprise a cylindrical shape. However, the disclosure contemplates other suitable shapes of he container 1. The container 1 may further comprise a cavity 14 having an inner surface 3. The cavity 14 may be defined by a deformable wall 4, a closed end 12 and an open end 16 scalable by a lid 2. The container 1 may be configured to hold a volume of anti-coagulated whole blood 5. In examples, a material of the inner surface 3 of the container 1 can be selected such that that the material activates the coagulation of the whole blood 5 during the centrifuge process by thrombin activation. For example, the material of the inner surface 3 may be selected from a group comprising polypropylene, polyethylene, polycarbonate, polyamide, acrylonitrile butadiene styrene, styrene, modified styrene, polyurethane and other polymer materials. In other examples, a material of the inner surface 3 can be selected such that the material is inactive in relation to the whole blood 5 and a separate object (not shown) made of a material that activates coagulation of the whole blood 5 may be added to the container 1. In yet further examples, the material of the container 1 can be selected to obtain a low friction between the inner surface 3 and any component of the blood 5, such as a material with a low protein binding capacity. The whole blood 5 may be subjected to a centrifugal force 6 acting downwards as illustrated. However, the disclosure also contemplates that the centrifugal force 6 is not limited to the direction shown. The centrifugal force 6 functions as a separation means because the components of the whole blood 5 have different densities and thus will respond differently to the centrifugal force 6.

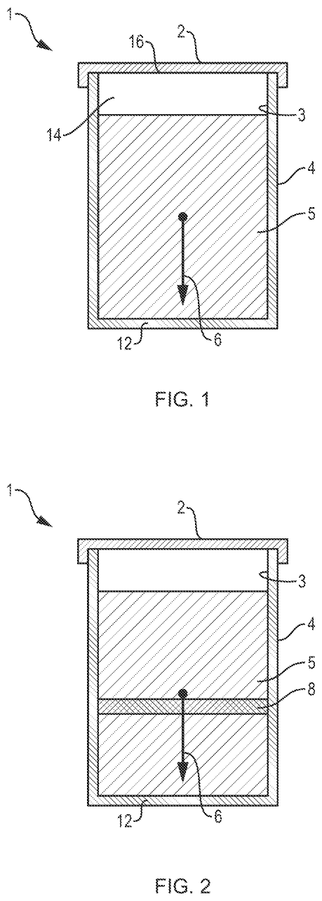

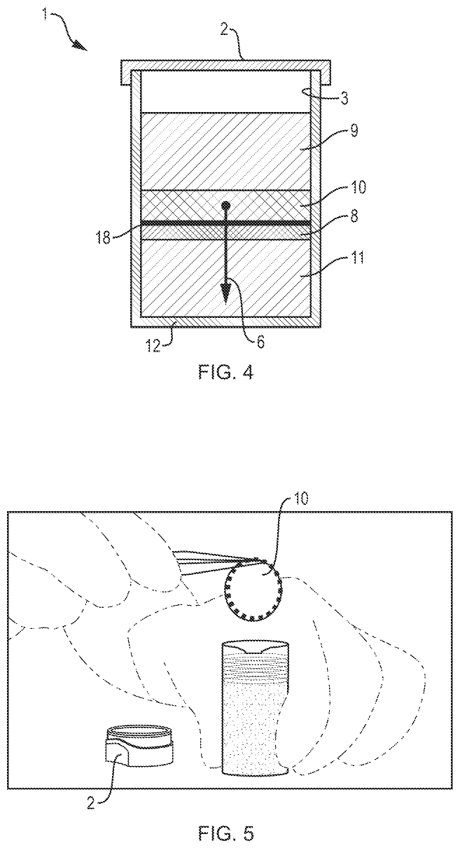

FIG. 2 illustrates another example of the container 1 according to an embodiment of this disclosure. As shown in FIG. 2, the container 1 may contain a compaction member 8, such as a fenestrated filter, whereby a bioactive implant 10 (FIG. 4) can be compacted against the compaction member 8. The compaction member 8 may have a circular shape, as shown.

However, the disclosure contemplates other suitable shapes of the compaction member 8. The compaction member 8 can be locked into the closed end 12 of the container 1 in the initial part of the centrifuge process. For example, the compaction member 8 may be fixed into the container 1 by deforming the wall 4. The compaction member 8 may then be released when the deformation of the wall 4 is released during the centrifuge process. The compaction member 8 can also be used to support the bioactive implant 10 during transport from the container 1.

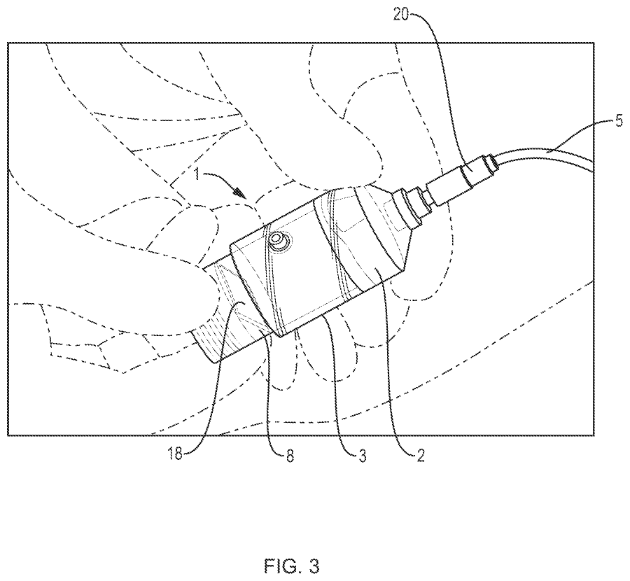

FIG. 3 illustrates another example of the container 1 according to an embodiment of this disclosure. As shown in FIG. 3, a collagen-based scaffold 18 may be placed within the container 1 on the compaction member 8 or elsewhere within the container 1 before the addition of the whole blood 5 to the container 1. In examples, the collagen-based scaffold 18 may be a sheet-like structure formed from reconstituted collagen material. For example, the collagen-based scaffold 18 may be a Regeneten™ BioInductive Implant (Smith & Nephew, Memphis, TN) derived from highly purified bovine Achilles tendon. The collage-based scaffold 18 may also be porous to allow filtering of the whole blood 5 during the compaction step. However, the disclosure contemplates other types of collagen-based scaffolds. Once the collagen-based scaffold 18 is placed onto the compaction member 8, the whole blood 5 may be added to the container 1 by means of an intravenous attachment 20 configured to draw the blood 5 directly from the patient into the container 1 through the sealed lid 2. Once the whole blood 5 has been added to the container 1, the whole blood 5 may be allowed to coagulate due to thrombin activation by the material of the inner surface 3.

FIG. 4 illustrates a method of creating the bioactive implant 10 during the centrifuge and coagulation process. As shown in FIG. 4, after the centrifugal force 6 has been acting for a sufficient period of time and with sufficient magnitude, the whole blood 5 may separate into a serum layer 9, the bioactive implant 10 (comprising PRP and leucocytes), and an erythrocytes layer 11 due to the differences in densities between the layers. The compaction member 8 may be released under centrifugal force and, provided that the density of the compaction member 8 is lower than that of the plasma, the compaction member 8 will be forced to the top of the plasma, thereby coupling the collagen-based scaffold 18 with the bioactive implant 10. Thus, the bioactive implant 10 will be suffused with collagen from the collagen-based scaffold 18. Further non-limiting examples of the centrifuge and coagulation process are described in U.S. Pat. No. 10,933,095 (Reapplix AS, Birkerod, Denmark) incorporated herein by reference. As shown in FIG. 5, after removing the lid 2, the bioactive implant 10 can be removed from the container 1 and used in a tissue repair.

The disclosure contemplates that other methods of augmenting the bioactive implant 10 inside the container 1 could be used. For example, the collagen-based scaffold 18 could be soaked in PRP prior to being placed on the compaction member 8. The scaffold could be made from other biologics, such as bone marrow aspirate, adipose, hyaluronic acid, stem cells, or allografts. In examples, rather than compacting the collagen-based scaffold 18 into the bioactive implant 10, the collagen-based scaffold 18 could be stitched to the bioactive implant 10 after removal from the container 1. In examples, the container 1 could also be configured to accept an external scaffold, such as an autograft.

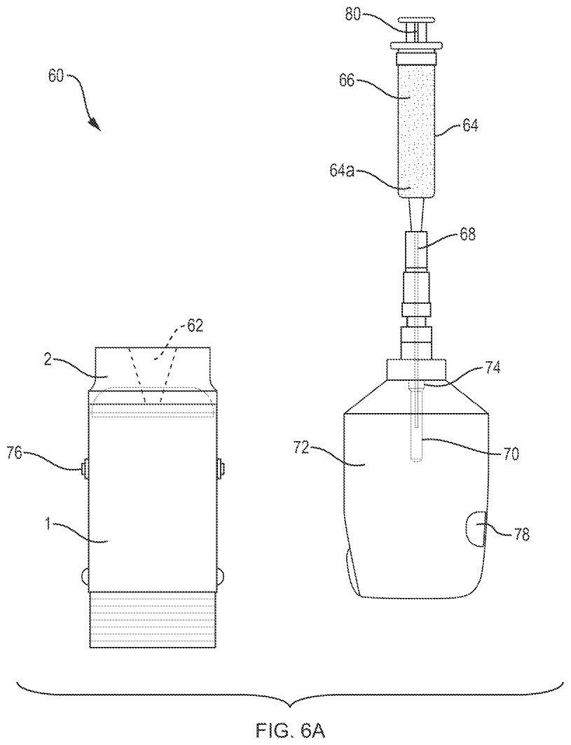

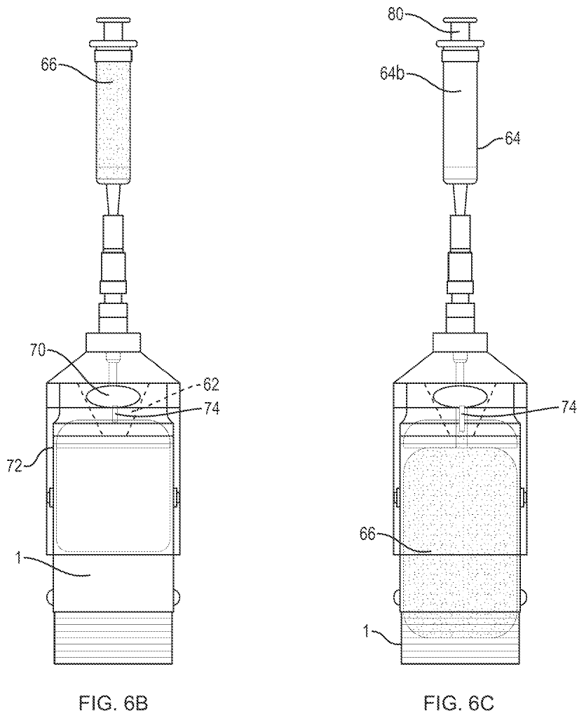

FIGS. 6A-6C illustrate an example of a system 60 for inserting a liquid biologic material into the container 1, either together with or instead of the collagen-based scaffold 18, for creating the bioactive implant 10. As shown in FIG. 6A, the system 60 may comprise the container 1 and the lid 2. The lid 2 may define a V-shaped opening 62 in communication with the interior of the container 1. The system 60 may also comprise a syringe 64 for containing the liquid biologic material 66 to be added to the container 1. In non-limiting examples, the liquid biologic material 66 may comprise one or more of bone marrow aspirate concentrate (BMAC), adipose tissue, hyaluronic acid (HA), placental tissue, stem cells, allografts, other biologic adjuncts, loose collagen fibers, or mixtures thereof. A needle cannula 68 may couple to a distal end 64a of the syringe 64. A retractable barrier 70 may further couple to a distal end 68a of the needle cannula 68. In examples, the retractable barrier 70 may be made from a flexible material, such as rubber, that is capable of crumpling under pressure. A connector 72 may couple to the needle cannula 68 such that the retractable barrier 70 extends into an interior of the connector 72. In examples, the connector 72 may have an inverted bowl or cup shape configured to be disposed over the lid 2 and the container 1 such that the barrier 70 enters the opening 62. A needle 74 in fluid communication with the interior of the syringe 64 may extend through the needle cannula 68 and partially into the barrier 70. In examples, an exterior surface of the container 1 may comprise one or more projections 76 for engaging corresponding slots 78 defined by the connector 72 to secure the connector 72 to the container 1. As shown in FIG. 6B, when the connector 72 is disposed over the opening 62 in the lid 2, the V-shape of the opening 62 may cause the barrier 70 to retract relative to the needle 74, exposing a distal end of the needle 74. As such, the needle 74 may be kept in a sterile state until the user is ready to deliver the liquid biologic material 66 into the container 1. As shown in FIG. 6C, once the needle 74 is exposed, the user may activate a plunger 80 at the proximal end 64b of the syringe 64 to deliver the liquid biologic material 66 into the container 1 before the centrifugation step.

The disclosure also contemplates that the barrier 70 could be disposed within the container 1 rather than attached to the needle cannula 68. The disclosure further contemplates that other types of syringes 64 may be configured to interact with the container 1. For example, the syringe 64 could be adapted to attach directly to the container 1. The disclosure further contemplates that the container 1 may contain a vacuum to aid in the injection of the liquid biologic material 66 into the container 1. A luer lock may be disposed between the syringe 64 and the container 1 to allow the container 1 to withstand injection backpressure. In other examples, the container 1 may include an outflow pressure release valve to allow for the liquid biologic material 66 to be added manually, without the need for a vacuum. These alternatives may advantageously provide additional versatility and customizability to the process of making the bioactive implant 10 by the user.

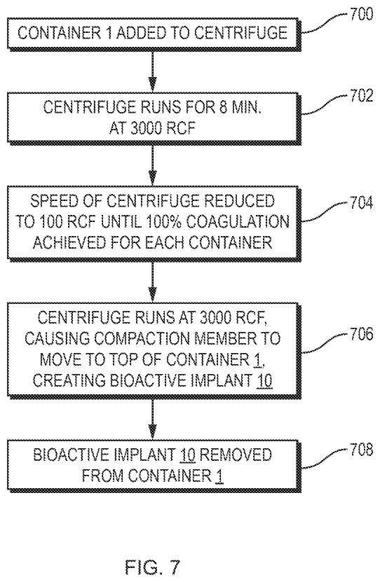

FIG. 7 is a flow-chart illustrating the steps of a non-limiting method of operating a centrifuge to creating the bioactive implant 10 using the system of FIGS. 6A-6C. In the example of FIG. 7, the centrifuge uses a “swinging bucket” rotor. However, the disclosure contemplates the use of centrifuges with other types of rotors, such as fixed-angle rotors. As shown in step 700 of FIG. 7, once the liquid biologic material 66 has been added to the container 1 and the patient's whole blood 5 has been collected, one or more containers 1 may be added to a centrifuge. If necessary, one or more counterweights can be added to the centrifuge to balance out an odd number of containers 1. During the centrifugation step 702, the centrifuge may run for approximately 8 minutes at 3000. Relative Centrifugal Force (RCF). During the coagulation step 704, the centrifuge speed may be reduced to 100 RCF. An optical sensor may be configured to detect coagulation by measuring the light transmission through the container 1. A display may be configured to show a user the coagulation progress (by percentage) for each container 1 until 100% coagulation is achieved. After the coagulation step 704, the rotation of the centrifuge may stop, and the containers 1 may automatically shift their position within the centrifuge before the compaction step 706 begins to release the compaction member 8 from its locked state within the container 1. During the compaction step 706, the centrifuge may again spin at 3000 RCF, causing the compaction member 8 to move toward the top of the container 1. A position of the compaction member 8 may be monitored by light transmission. Once the compaction member 8 reaches a preselected position within the container 1, the centrifuge may stop. In the final step 708, the bioactive implant 10 may be removed from the container 10. The disclosure also contemplates that other suitable centrifuge processes (i.e., RCF, stages, timing, etc.) could be used to create the bioactive implant 10. Furthermore, the compaction member 8 may have different densities depending on the desired properties of the bioactive implant 10, or the liquid biologic material 66 of choice.

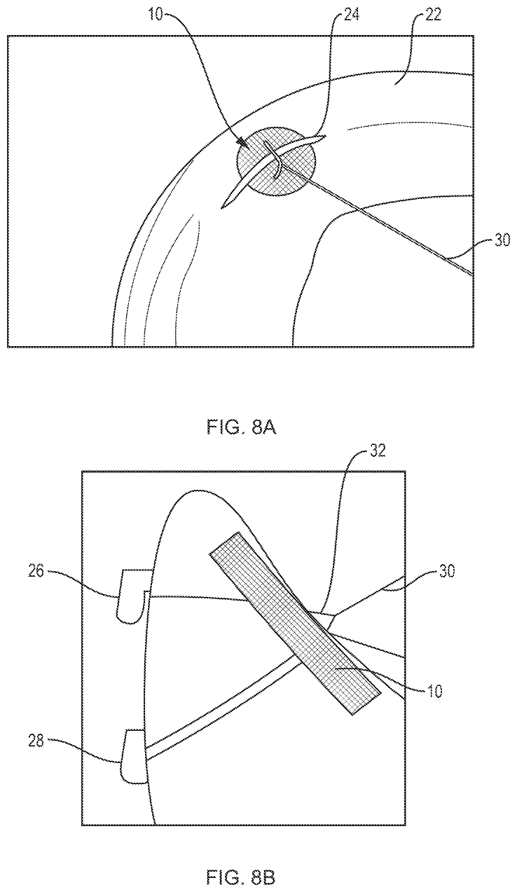

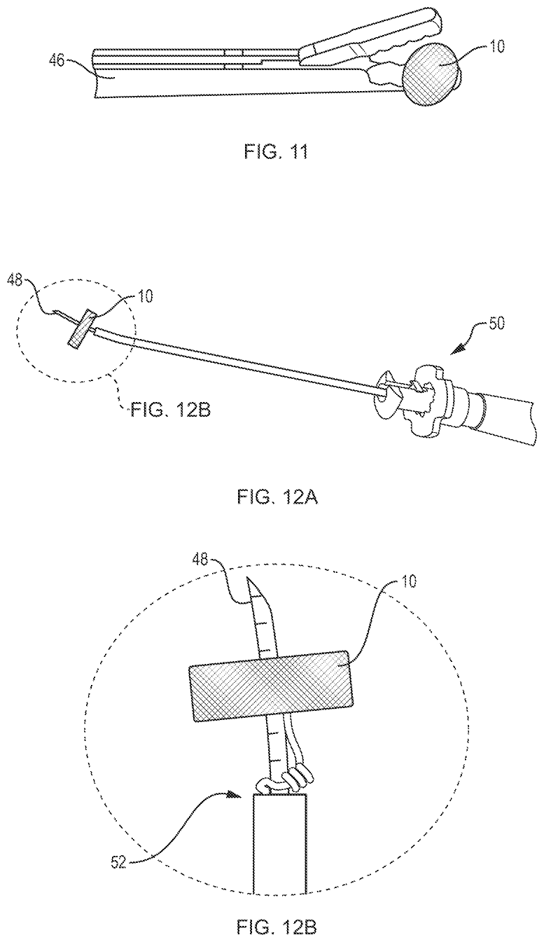

FIGS. 8A and 8B illustrate a non-limiting method of using the bioactive implant 10 in a tissue repair of this disclosure, such as a repair of the meniscus 22. As shown in FIG. 8A, the bioactive implant 10 may be placed in contact with a wound or tear 24 in the meniscus 22. A meniscal repair device 50 (FIG. 12A) can then be used to pierce the implant 10 and fix it in place. For example, as shown in FIG. 8B, the meniscal repair device can fix the implant 10 in place using a first anchor 26 and a second anchor 28 connected by a suture 30. Once the anchors 26, 28 are in place in the repair, the suture 30 can be tensioned such that a slidable knot 32 in the suture 30 is reduced toward the implant 10, compressing the implant 10 against the tear 24.

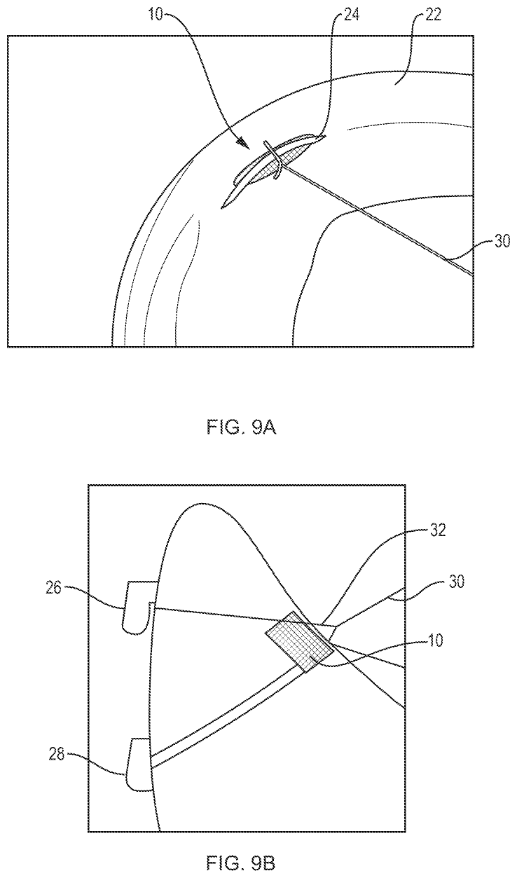

FIGS. 9A and 9B illustrate another non-limiting method of using the bioactive implant 10 in a tissue repair of this disclosure, such as the repair of the meniscus 22. As shown in FIG. 9A, using a tissue grasper 46 (FIG. 11), the bioactive implant 10 may be placed in contact with the tear 24 in the meniscus 22. The meniscal repair device 50 can then be used to place the first anchor 26 and the second anchor 28 connected by the suture 30 over the implant 10 without piercing the implant 10. Once the anchors 26, 28 are in place in the repair, the suture 30 can be tensioned such that the knot 32 is reduced toward the implant 10, fixing the implant 10 in place against the tear 24.

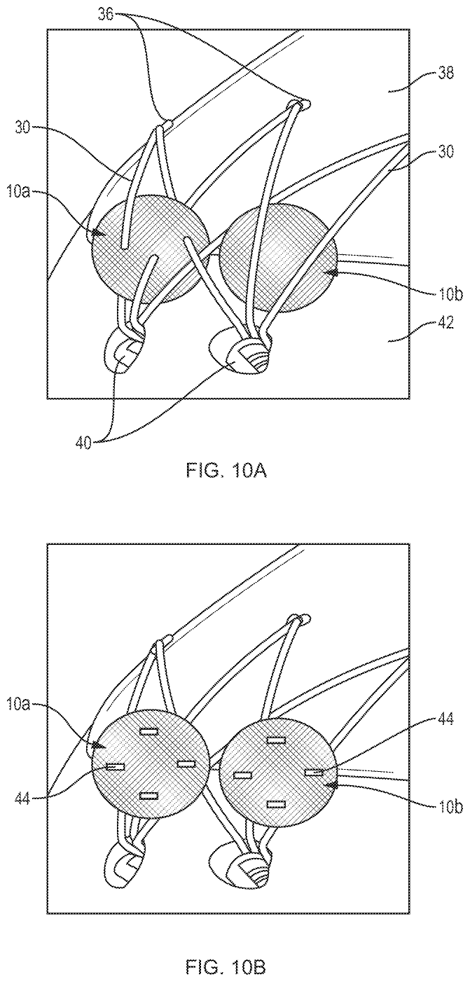

FIGS. 10A and 10B illustrate other non-limiting methods of using the bioactive implants 10a, 10b in a tissue repair of this disclosure, such as a repair of the rotator cuff (not shown). As shown in FIG. 10A, the bioactive implants 10a, 10b may be placed in contact with a wound or tear in the rotator cuff. A surgical repair system can then be used to pierce the implant 10a and fix it in place. For example, the repair system can fix the implant 10a in place with anchors 36 placed in the scapula 38 connected by suture 30 to anchors 40 placed in the humeral head 42. Alternatively, the suture 30 may be routed over the implant 10b without piercing the implant 10b. As shown in FIG. 10B, the implants 10a, 10b may also be placed underneath the rotator cuff in the area of the wound or tear. The surgeon may then insert staples 44 (such as tendon staples) through the rotator cuff and through the implants 10a, 10b, to hold the implants 10a, 10b in place.

The disclosure also contemplates that the implant 10 may be used in other types of tissue repairs, such as a hip capsule or Achilles tendon repair. Other non-limiting examples of uses of the implant 10 include tendon wraps, labral repair adjuncts, quad tendon harvest site repair, foot and ankle orthopedic incision sites, epicondylitis, biceps repair, hip gluteus repair, piriformis, iliopsoas, medial collateral ligament repair of the knee, patellar tendon repair, and other sports medicine applications. The implant 10 can also be glued over cartilage defects.

In all tissue repairs using the bioactive implant 10 described above, the implant 10 can be introduced into the repair in several ways. For example, arthroscopic graspers, such as the grasper 46 shown in FIG. 11, can be used to hold the implant 10 and deliver it into the joint space. Notably, graspers without roughened teeth may be beneficial for delicate handling of the implant 10. In other examples, not shown, suture extending from a suture anchor may be used as a ‘zip line’ to guide the implant 10 into the joint. This may be accomplished by threading the suture through the middle of the implant 10 and pushing the implant 10 down the suture using an instrument such as a knot pusher.

In further examples, not shown, a suture extending from an anchor may have two free ends extending outside the anchor body. One limb of the suture may be passed through the implant 10, with a knot, such as a mulberry knot, being tied proximal to the implant 10. The implant 10 can then be shuttled into the joint by pulling on the other free limb of the suture. The mulberry knot may act as a backstop to pull the implant 10 into the joint. Similar techniques can use a mulberry knot and an independent suture that is passed through the tissue repair point of interest. It is also possible to use two suture anchors, with a mulberry knot tied between a limb from each of their respective sutures, and using the two free limbs (one from each anchor) to shuttle the implant 10 in.

In other examples, the implant 10 may be pierced with a needle 48 of a meniscal repair device 50, such as the meniscal repair device 50 shown in FIGS. 12A and 12B. A first anchor residing in the needle 48 may be inserted through the meniscus, distal to the implant 10. A second anchor residing in the needle 48 and attached to the first anchor by a suture may be inserted into the meniscus proximal to the implant 10 to fix the implant 10 in place. A tube 52 for limiting the insertion depth of the needle 48 may act as a backstop to the implant 10.

In all methods of tissue repair described above, the implant 10 and any instrumentation can be introduced into a joint space through a slotted or regular cannula, or introduced percutaneously (i.e., no cannula). The implant 10 can also be placed in the distal end of a small diameter cannula for introduction into the joint space and pushed out with the use of an obturator in the desired location.

While the disclosure particularly shows and describes preferred examples, those skilled in the art will understand that various changes in form and details may exist without departing from the spirit and scope of the present application as defined by the appended claims. The scope of this present application intends to cover such variations. As such, the foregoing description of examples of the present application does not intend to limit the full scope conveyed by the appended claims.

We claim:

1. A method of preparing a bioactive implant for application to a wound or tear in a tissue of a patient, the method comprising:
adding a volume of the patient's whole blood into a container, the container including a collagen-based scaffold disposed within the container and a movable member locked within the container;
applying a centrifugal force to the container sufficient to separate the bioactive implant from a layer of serum and a layer of erythrocytes; and
releasing the moveable member such that the moveable member moves though the container under the centrifugal force to couple the collagen-based scaffold with the bioactive implant, whereby the bioactive implant is suffused with collagen from the collagen-based scaffold.

2. The method of claim 1, wherein the moveable member is a filter.

3. The method of claim 1, wherein the bioactive implant comprises platelet rich plasma.

4. The method of claim 1, wherein the method further comprises, before applying the centrifugal force, adding to the container one or more biologic materials selected from a group comprising: bone marrow aspirate concentrate (BMAC), adipose tissue, hyaluronic acid (HA), placental tissue, stem cells, allografts, other biologic adjuncts, loose collagen, or mixtures thereof.

5. The method of claim 1, further comprising, after injecting the volume of the patient's whole blood into the container, allowing the whole blood to coagulate.

6. The method of claim 1, wherein the collagen-based scaffold is derived from highly purified bovine Achilles tendon.

7. The method of claim 1, further comprising removing the bioactive implant from the container.

8. A bioactive implant formed by the method of claim 1.

9. A system for inserting a liquid biologic material into a container, the system comprising:
a syringe containing the liquid biologic material;
a needle cannula coupled to a distal end of the syringe;
a retractable barrier coupled to a distal end of the needle cannula;
a connector coupled to the needle cannula such that the retractable barrier extends into an interior of the connector, the connector configured to be disposed over an opening of a container; and
a needle in fluid communication with the syringe extending through the needle cannula and into the barrier;
wherein, when the connector is disposed over the opening in the container, the opening causes the retractable barrier to retract, exposing a distal end of the needle to deliver the liquid biologic material into the container.

10. The system of claim 9, wherein the container comprises a vacuum.

11. The system of claim 9, wherein the opening is a V-shaped opening defined through a lid of the container.

12. A method of tissue repair, the method comprising:
placing a bioactive implant in contact with an area in need of repair in a tissue of a patient; and
securing the bioactive implant to an area adjacent the area in need of repair with at least one securing device extending through or around the bioactive implant;
wherein the bioactive implant is formed by a process of:
injecting a volume of the patient's whole blood into a container; and
applying a centrifugal force to the container sufficient to separate the bioactive implant from a layer of serum and a layer of erythrocytes.

13. The method of claim 12, wherein bioactive implant is coupled to a collagen-based scaffold.

14. The method of claim 12, wherein the tissue is a tendon, ligament or other soft tissue.

15. The method of claim 12, wherein the tissue is a rotator cuff or a meniscus.

16. The method of claim 12, wherein the securing device is a suture.

17. The method of claim 12, wherein the securing device is a staple.

18. The method of claim 12, wherein the area in need of repair is a wound.

19. The method of claim 12, wherein the area in need of repair is a tear.
